(19) United States
(12) Patent Application Publication
(10) Pub. No.: US 2020/0080156 A1
(43) Pub. Date: Mar. 12, 2020

(54) PROSTATIC CANCER MARKER, PLXNA1, AND APPLICATION THEREOF

(71) Applicant: SHANGHAI CHANGHAI HOSPITAL, Shanghai (CN)

(72) Inventors: Yinghao SUN, Shanghai (CN); Shancheng REN, Shanghai (CN); Yasheng ZHU, Shanghai (CN); Xiaolei SHI, Shanghai (CN); Yue YANG, Shanghai (CN)

(73) Assignee: SHANGHAI CHANGHAI HOSPITAL, Shanghai (CN)

(21) Appl. No.: 16/481,331

(22) PCT Filed: Dec. 15, 2017
(86) PCT No.: PCT/CN2017/116368

§ 371 Date: Nov. 19, 2019

(30) Foreign Application Priority Data
Jan. 26, 2017 (CN) 2017100573079

(51) Int. Cl. C12Q 1/6886; G01N 33/50; G01N 33/574; C12N 15/113

(52) U.S. Cl. CPC C12Q 1/6886; G01N 33/5011; G01N 33/57434; C12N 15/113; C12N 2310/531; C12Q 2600/158; C12Q 2600/112; C12N 2310/14; C12Q 2600/118

(57) ABSTRACT

The present invention provides an application of PLXNA1 as a biomarker for predicting the occurrence, development, and prognosis of prostate cancer and for distinguishing prostate cancer being progressive and with high malignancy. By detecting a change in a PLXNA1 DNA copy number, and expression levels of mRNA and a protein encoded thereby, a diagnosis and prognosis of prostate cancer are performed. The present invention also provides an application of PLXNA1 as a therapeutic target for prostate cancer. The present invention also provides a kit for diagnosing and predicting prostate cancer.

### REFERENCE TO SEQUENCE LISTING SUBMITTED VIA EFS-WEB

This application includes an electronically submitted sequence listing in .txt format. The .txt file contains a sequence listing entitled “6075-0120PUS1_ST25.txt” created on Nov. 4, 2019 and is 28,945 bytes in size. The sequence listing contained in this .txt file is part of the specification and is hereby incorporated by reference herein in its entirety.—

### TECHNICAL FIELD

The present invention relates to cancer diagnosis field, particularly to PLXNA1 being as a biomarker for distinguishing prostate cancer being progressive and with high malignancy, its application, and a kit thereof.

### BACKGROUND OF THE INVENTION

Prostate Cancer (PCa) is known as a type of malignant tumor that poses threat to men. The incidence and mortality rates are the highest for men aged around 70, which put prostate cancer a second place on the ranking of global cancer incidence rates and a sixth place for mortality rates. In 2016, the United States reported 180,890 new cases and 26,120 deaths. Nevertheless, significant racial disparities have been observed in prostate cancer. Take the United States as an example, the incidence rate in African Americans doubles that in White Americans. The incidence rate in Asia is much lower than in Western countries. It is therefore prompted to perform genome analysis for each different ethics.

Prostate cancer is known to exhibit individual differences, and the response of individual patients to the same treatment regimen may vary dramatically. Some patients can survive for more than 10 years after cancer diagnosis, but some others only for 2 to 3 years. Therefore, researches focused on prostate cancer genes, gene phenotypic heterogeneity, and changes in signaling pathways in prostate cancer progression have great deal of importance. In recent years, with the development of targeted sequencing, copy number variation and whole genome sequencing technology, major breakthrough has been made with the prostate cancer gene landscape researches. A large number of studies have revealed some genomic changes related to prostate cancer, such as: the most common gene fusion of TMPRSS2-ERG, copy number increase at 8q and proto-oncogene DNA recombination (Chromoplexy), etc. However, the results of these genomic changes remain unclear. Exome sequencing technology has revealed some changes of the specific gene in the coding region, and found some mutant genes with high frequency such as SPOP, FOXA1, TP53, PTEN and other genome changes in PIK3CA/B, ZBTB16/PLZF and AR. Recently, The Cancer Genome Atlas (TCGA) in the United States also performed a more comprehensive genome analysis of 333 patients with prostate cancer (mostly white men). It can be understood that many genome researches on prostate cancer for the western population have been carried out and progress has been made. On the other hand, in recent years, although the incidence of prostate cancer has increased significantly in medical and economically developed cities such as Beijing, Shanghai and Hong Kong, the genome researches on prostate cancer for Asian population is still in its infancy. Therefore, it is urgent to carry out corresponding genome research to further comprehend the molecular pathology of prostate cancer in order to better guide clinical work.

Prostate cancer, a type of malignant tumor exhibiting up until now the highest racial/ethnic disparities among all the cancers, is differently characterized within Chinese patients, whose PSA value at the time of prostate cancer diagnosis is higher than that of western people and score higher under the Gleason Score grading system. Besides, unlike as in Western countries, Chinese patients become aware of their illness at a late time when they could already have the cancer partially advanced or spread to other parts of the body at the time of diagnosis. Patients with advanced-stage prostate cancer make up the big portion of all cases, and as many as about 60% of patients have developed local progression or metastatic prostate cancer at the time of diagnosis.

It is well known that epithelial-to-mesenchymal transition (EMT) in tumor progression is significant in tumor metastasis. There are many mechanism studies have been focused on the EMT process. The totipotency of tumor cells is one of the important mechanisms to promote the malignant progression of tumors. Tumor-associated EMT processes and some cancer stem cell-associated molecules are important ways to conduct evaluation of cancer prognosis for patients and provide possible therapeutic targets.

Therefore, it is particularly important to confirm a mechanism that promotes the malignant progression of prostate cancer, especially one that may differ from the known mechanism in western population, and to find molecules that are clinically relevant to the prognosis of prostate cancer patients. At present, many detection kits for evaluating the prognosis of prostate cancer are available around the world, such commercialized products as Prolaris (46 genes), Decipher (22 genes) and Oncotype DX (17 genes), totally covering 85 genes, with Prolaris and Decipher sharing to use one common gene. None of the any products have been used in the samples from Chinese population. It can be understood there still calls for an ideal biomarker for molecular classification for prostate.

Thus, it is important to find a suitable biomarker for molecular typing especially for the Chinese population, that should have been validated with samples from Chinese and non-Chinese population, which will be useful for evaluating prostate cancer prognosis.

The present invention, by means of high-throughput sequencing combined with cell biology experiments, found that the expression of a molecule of the Axon Guidance signaling pathway, PLXNA1, is increased in prostate cancer tissues and particularly increased in prostate cancer with high Gleason score. The present invention further explored the biological function of the gene during the progression in prostate cancer. The present invention verified by a number of major databases, with the survival prognosis analysis of diverse population from within and out of China, confirmed that PLXNA1 can be used as an important biomarker in the prognosis of prostate cancer.

### SUMMARY OF THE INVENTION

According to a first aspect of the invention, there is provided an application of PLXNA1 as a biomarker for predicting the occurrence, development and prognosis of prostate cancer.

PLXNA1 (Plexin A1), as a receptor for semaphorin with signal transmembrane, is a component of the Axon guidance signaling pathway. PLXNA1 plays an important role in axons of neurons, causing aggregation and rejection of endothelial cells, regulating the function of immune cells and osteoclasts, through a series of intracellular signals pathways. At present, there are few studies on PLXNA1 in tumors, and only a few reports on gastric cancer, malignant pleural mesothelioma, and rhabdomyosarcoma.

The nucleotide sequence of PLXNA1 is shown as SEQ ID No. 1. The protein sequence encoded by the PLXNA1 gene is shown as SEQ ID No. 2.

In the present invention, PLXNA1 DNA, mRNA or a protein encoded thereby could be used as a prostate cancer biomarker for predicting the occurrence, development and prognosis of prostate cancer. By detecting a change in a PLXNA1 DNA copy number, and expression level of mRNA and a protein encoded thereby, a prediction of prostate cancer is performed.

In the present invention, sequences having homology of at least 80%, 85%, 90%, 95%, or 99% to the PLXNA1 DNA, also could be used as prostate cancer biomarkers.

In the present invention, sequences having homology of at least 80%, 85%, 90%, 95%, or 99% to the PLXNA1 mRNA, also could be used as prostate cancer biomarkers.

In the present invention, sequences having homology of at least 80%, 85%, 90%, 95%, or 99% to the PLXNA1 protein, also could be used as prostate cancer biomarkers.

Provided in the present invention is a use of a reagent for detecting of PLXNA1 in the preparation of a product for predicting the occurrence, development, and prognosis of prostate cancer, or, for distinguishing prostate cancer being progressive and with high malignancy.

Also provided in the present invention is an in vitro diagnostic product for prostate cancer, wherein the in vitro diagnostic product comprises a reagent for specific detection of PLXNA1 DNA, and/or a reagent for specific detection of PLXNA1 mRNA, and/or a reagent for specific detection of protein encoded by the PLXNA1 gene; wherein the in vitro diagnostic product comprises kit, gene chip, or solid support; solid support includes arrays, microarrays, or protein arrays.

Also provided in the present invention is a gene chip for prostate cancer by detecting PLXNA1, wherein the gene chip includes a reagent for detecting PLXNA1.

Also provided in the present invention is a kit for prostate cancer by detecting PLXNA1, wherein the kit includes a reagent for detecting PLXNA1 The kit is highly specific for detecting PLXNA1 while also is easy to use.

Also provided in the present invention is a use of PLXNA1 in the preparation of pharmaceuticals for suppressing the proliferation, metastasis, invasion of prostate cancer cells.

Also provided in the present invention is a use of PLXNA1 in the preparation of pharmaceuticals for suppressing the growth, metastasis of prostate tumors.

Also provided in the present invention is a use of PLXNA1 in the preparation of pharmaceuticals for suppressing the expression of interstitial cell marker vimentin.

Also provided in the present invention is a use of PLXNA1 in the preparation of pharmaceuticals for suppressing the expression of N-cadherin.

Also provided in the present invention is a use of PLXNA1 in the preparation of pharmaceuticals for suppressing the expression of fibronectin.

Also provided in the present invention is a use of PLXNA1 in the preparation of pharmaceuticals for suppressing the expression of stem cell markers such as CD44, CD133, CD49f, OCT4, NANOG, SOX2 and LIN28B, and neuroendocrine cell markers such as CgA, SYP and FOXA2.

Also provided in the present invention is a use of PLXNA1 in the preparation of pharmaceuticals for promoting the expression of epithelial cell marker E-cadherin.

Also provided in the present invention is an application of PLXNA1 as a therapeutic target for prostate cancer.

Also provided in the present invention is a use of an inhibitor of PLXNA1 in the preparation of pharmaceuticals for treating prostate cancer, wherein the inhibitor is selected from the group consisting of any of inorganic substance, an organic substance, a nucleic acid molecule, a receptor, an antagonist, a blocker, or the like, capable of suppressing the increase in copy number of the PLXNA1 DNA, or suppressing the transcription or expression of PLXNA1 DNA, mRNA or a protein encoded thereby, or suppressing the activity of the protein encoded by the PLXNA1 gene.

Also provided in the present invention is a use of a change in content of PLXNA1 in predicting the occurrence, development, and prognosis of prostate cancer.

Also provided in the present invention is a use of a change in content of PLXNA1 in distinguishing prostate cancer being progressive and with high malignancy.

Also provided in the present invention is a method for prognosis of prostate cancer in patients, comprising the following steps: detecting the DNA copy numbers of PLXNA1, or the expression levels of mRNA or the protein encoded thereby in pathology samples of prostate cancer; if DNA copy number of PLXNA1 is amplified or overexpressed of the patient compared to normal population, or if the expression level of mRNA or the protein encoded thereby of the patients increases, then the patient is classified into high expression group, indicating deteriorating, highly invasive of tumor, poor prognosis for survival, likelihood of biochemical recurrence, distant metastasis or disease progression or death; whereas the patients is classified into low expression group.

The high expression group is referred to as those patients, as opposed to the normal controls, who exhibit over amplification in copy number of the PLXNA1 DNA or over expression of the PLXNA1 gene (that is, there are statistically significances in amplification in copy number of the PLXNA1 DNA or over expression of the PLXNA1, relative to normal control, for example P<0.05 or P<0.01, etc); or the patients exhibit an increase in the expression levels of mRNA or a protein encoded thereby, indicative of a statistically significance relative to normal control, for example P<0.05 or P<0.01, etc. (take the DAB coloration in immunohistochemical staining for example, semi-quantitative assays were performed for comprehensive staining intensity and the proportion of positive cells at high magnification; staining intensity scoring criteria: low expression—no coloration or light yellow, high expression—pale brown or dark yellow brown).

The expression of PLXNA1 in prostate cancer tissues was detected by immunohistochemistry. Pathological examinations were performed. The patients were divided into high expression group and low expression (including no expression or negative) group according to the expression of PLXNA1. It was shown that, the malignant degree of prostate cancer of the high expression group was significantly higher than that of the low expression group; and the occurrence of biochemical recurrence and distant metastasis post radical prostatectomy of the high expression group were significantly earlier than that of the low expression group; and the overall survival time of the patients in high expression group was significantly shorter than that of the low expression group. At the same time, through bioinformatics analysis, it was showed that in the present invention, for patients with prostate cancer who exhibited abnormal increase in PLXNA1 DNA copy number, the malignancy degree of the tumors were significantly higher; the occurrence of biochemical recurrence and distant metastasis post radical prostatectomy were significantly earlier than that of the patients who exhibited normal in PLXNA1 DNA copy number; and the overall survival time was significantly shorter than that of the patients who exhibited normal in PLXNA1 DNA copy number. Therefore, for high expression group, during clinical diagnosis and treatment process more frequent follow-up visits and observations are suggested. The detection of the expression of PLXNA1 in patients is considered helpful for the evaluation of prognosis of patients, and for the guidance for patients in clinical diagnosis and treatment decisions.

To clarify the role of PLXNA1 in prostate cancer, the present invention detected the effects of the si-RNA-mediated PLXNA1 knockdown on cell phenotype, and found that the invasive ability (FIG. 1a) and migration ability (FIG. 1b) of prostate cancer cells were significantly suppressed after the knockdown of PLXNA1 expression by si-RNA.

It was found in the present invention that, cell proliferation, invasion and migration abilities in different types of prostate cancer cell lines that were examined (including C4-2, PC-3 and LNCaP), were significantly suppressed by means of knockdown of PLXNA1 expression by si-RNA (FIG. 1c-e). Furthermore, it was shown that, in the prostate cancer cell line DU145, over expression of PLXNA1 significantly have significantly enhanced cell proliferation (FIG. 1f), invasion and migration (FIG. 1g).

The same phenomenon was also observed in prostate cancer cell lines with high expression of PLXNA1 in progressive prostate cancer cell lines (ARCaP_{M} cell line and LNCaP^{RANKL} cell line) (FIG. 2), that is, cell proliferation, invasion and migration were promoted. Both cell lines have the properties of interstitial cell and are easy to metastasis to bones and soft tissues (FIG. 2).

The phenotype of epithelial-to-mesenchymal transition, totipotency and neuroendocrine properties are all associated with metastatic castration-resistant prostate cancer. The inventors of this invention made the hypothesis that whether knockdown of PLXNA1 may affect the expression of these progressive phenotypic markers. After performing knockdown of PLXNA1 in LNCaP^{RANKL} cell line and ARCaPM cell line, the expression of interstitial cell marker vimentin, N-cadherin and fibronectin decreased, while the expression of epithelial cell marker E-cadherin increased (FIG. 3a). Moreover, the inventors of this invention observed that the expression of stem cell markers CD44, CD133, CD49f, OCT4, NANOG, SOX2 and LIN28B and neuroendocrine cell markers CgA, SYP and FOXA2 were suppressed (FIG. 3a). Similarly, in prostate cancer cell lines with abnormally high expression of PLXNA1, the phenotype of epithelial-to-mesenchymal transition, totipotency and neuroendocrine were increased, indicating that the higher the degree of the tumor malignancy, the easier of recurrence, metastasis, and drug resistance (FIG. 3b).

The present invention then evaluated the effect of PLXNA1 knockdown on cell growth using an in vivo tumor-bearing nude mouse model. The size and weight of tumor were significantly reduced in the nude mice with shRNA-mediated PLXNA1 stable knockdown compared to the control group (FIG. 4a). Correspondingly, the proliferation of prostate cancer cell lines with PLXNA1 overexpressing in tumor-bearing nude mice was significantly faster than that in control group (FIG. 4a). Immunohistochemical staining analysis of the tumor in tumor-bearing mice showed that, the expression of Ki-67 (one of the markers of cell proliferation) decreased significantly after PLXNA1 knockdown, indicating that cell proliferation rate decreased significantly (FIG. 4b). In addition, compared with control group, phenotype reversal of the epithelial-to-mesenchymal transition was observed in the tumors with PLXNA1 knockdown, accompanied with the results of: increased expression of E-cadherin (E-cad) expression, increased expression of N-cadherin (N-cad), vimentin and fibronectin; along with suppressed expression of neuroendocrine phenotype, accompanied with the results of: weakened staining of CgA and Syp (FIG. 4b). The above was consistent with the phenomenon observed in malignant prostate cancer cell lines (FIG. 2). Therefore, through the above relevant functional experiments, it was found that PLXNA1 plays an important role in the regulation of cell proliferation, invasion and metastasis, suggesting that high expression of PLXNA1 is associated with high degree of malignancy of tumor, and a change in the expression of PLXNA1 may be used to predict the occurrence, development, and prognosis of prostate cancer.

In order to evaluate the clinical significance of PLXNA1 in the occurrence, development of human prostate cancer, the present invention first studied and explored the possible relationship between the expression of PLXNA1 and the degree of malignancy of tumor. Through data analysis utilizing the existing clinical database, the inventors of this invention found that the expression of PLXNA1 was significantly higher in metastatic prostate cancer than in non-metastatic prostate cancer (FIG. 5a-d), and the expression of PLXNA1 was significantly higher in prostate cancer tissues than in the corresponding normal tissues (FIG. 5e). In addition, up-regulation of PLXNA1 was associated with high PSA level (FIG. 5f, g) and high-grade clinical stage (FIG. 5h).

Through immunostaining of PLXNA1 in a separate cohort of 87 patients with primary prostate cancer from Shanghai Changhai Hospital, it was found that high expression of PLXNA1 was associated with high Gleason score, progressive tumor stage, and fast biochemical recurrence after radical prostatectomy (FIG. 6a). The present studies showed that increased expression of PLXNA1 is associated with poor prognosis. In the other two independent cohorts of prostate cancer patients (data respectively from Glinsky 2004 and TCGA), the inventors of this invention also observed that high expression of PLXNA1 mRNA was associated with fast biochemical recurrence after radical surgery of prostate cancer in patients (FIG. 6b, c).

To further clarify the role of PLXNA1 in prognosis of prostate cancer, immunostaining of PLXNA1 was conducted by immunohistochemistry assay to detect the expression of PLXNA1 in two groups of independent samples of tissue microarray (one group from China Prostate Cancer Consortium (CPCC), n=419, 4 institutions, 10-year follow-up; another group from Massachusetts General Hospital (MGH), n=213, 20-year follow-up). It was found that high expression of PLXNA1 was not only associated with biochemical recurrence but also significantly with non-metastasis survival and overall survival (FIG. 7a, b). It is worth noting that multivariate and univariate regression analysis showed that increased expression of PLXNA1 was an independent factor predicting biochemical recurrence, non-metastasis survival and overall survival, prognosis in patients with prostate cancer (The risk ratios of CPCC group in patients with high expression of PLXNA1 for biochemical recurrence, non-metastasis survival, and overall survival were 2.75, 3.63, and 2.66 respectively. The risk ratios of Massachusetts General Hospital group in patients with high expression of PLXNA1 for biochemical recurrence, non-metastasis survival, and overall survival were 1.67, 2.31 and 2.24 respectively) (FIG. 7a, b). The above experimental data and analysis results suggest that PLXNA1 can be used as a novel marker to evaluate the degree of malignancy of prostate cancer.

Since the increase in the expression of PLXNA1 is related to the variation of copy number of DNA, as mentioned above, the increase in the expression of PLXNA1 is associated with a higher degree of malignancy of prostate cancer. The present inventors further investigated whether the increase in the copy number of PLXNA1 is directly related to the recurrence of prostate cancer.

Analysis showed that the frequency of increase in PLXNA1 copy number was very high in metastatic prostate cancer (FIG. 7c). Furthermore, by analyzing a cohort of TCGA data of patients with prostate cancers, the inventors of this invention found that the probability of biochemical recurrence in prostate cancer patients with increased PLXNA1 copy number was significantly higher than in patients without increased PLXNA1 copy number (TCGA data, FIG. 7d). Therefore, amplification of copy number and overexpression of PLXNA1 DNA could promote the growth and progression of prostate cancer, and they are associated with higher degree of malignancy and poor prognosis of prostate cancer, and therefore could be used as a biomarker for prognosis of prostate cancer.

The present invention is advantageous in that: The present invention discloses a novel biomarker PLXNA1 for prostate cancer; the change in the expression level of the protein encoded by the PLXNA1 gene in the prostate cancer tissue and the change of the copy number of PLXNA1 DNA can be used as a significant marker in evaluating the degree of malignancy of prostate cancer in progressive stage, predicting the occurrence, development, and prognosis of prostate cancer. The role of the novel biomarker PLXNA1 for evaluating the prognosis of prostate cancer patients in the present invention has been verified by bioinformatics analysis, clinical specimen detection and data analysis, and has been confirmed in a plurality of samples from Chinese and European and American populations. By single factor and multivariate regression analysis, it was found that, compared with some existing indicators for evaluating the prognosis of patients (such as Gleason score, etc.), PLXNA1 of the present invention is an independent risk factor for evaluation of the occurrence, development, and prognosis of prostate cancer, distinguishing prostate cancer being progressive and with high malignancy.

### BRIEF DESCRIPTION OF THE DRAWINGS

FIG. 1 shows that, after knockdown of PLXNA1 by si-RNA in prostate cancer cells C4-2, PC-3, LNCaP, invasion capacity (a, d) and migration capacity (b, e) of prostate cancer cells were significantly reduced; after knockdown of PLXNA1 by si-RNA in prostate cancer cells C4-2, PC-3, LNCaP, the proliferative capacity of prostate cancer cells was significantly reduced (c); after overexpression of PLXNA1 in prostate cancer DU145 cell line by plasmid, the capacities of proliferation, migration and invasion of prostate cancer cells were significantly improved (f, g).

FIG. 2 shows that, improved properties of proliferation, invasion and migration of prostate cancer cells were observed in progressive prostate cancer cell lines with high expression of PLXNA1 (ARCaPM cell line and LNCaP^{RANKL} cell line).

FIG. 3 shows that, prostate cancer epithelial-to-mesenchymal transition EMT related indicators, and stem cell and neuroendocrine related indicators were changed after knockdown of PLXNA1 in LNCaP^{RANKL} cell line and ARCaPM cell line (a); in prostate cancer cell line with abnormally high expression of PLXNA1, the phenotypes of epithelial-to-mesenchymal transition, totipotency and neuroendocrine were all changed (b).

FIG. 4 shows that, proliferation of prostate cancer cell lines with PLXNA1 knockdown in tumor-bearing nude mice is significantly slower than that of the control group, whereas proliferation of prostate cancer cell lines with PLXNA1 overexpressing in tumor-bearing nude mice is significantly faster than the control group (a); changes in indicator related to epithelial-to-mesenchymal transition, totipotency and neuroendocrines in PC-3 prostate cancer cell lines with PLXNA1 knockdown and control cell lines (b).

FIG. 5 shows that, the expression level of PLXNA1 in metastasis prostate cancer is significantly higher than that of non-metastatic prostate cancer (a-d); the expression level of PLXNA1 in prostate cancer tissues is significantly higher than that in normal control tissues (e); up-regulation of PLXNA1 is associated with increased PSA level (f, g); and is associated with high clinical stage prostate cancer (h).

FIG. 6 shows that, high expression of PLXNA1 is associated with high Gleason score, progressive tumor stage, and fast biochemical recurrence after radical surgery (a); it is found that high expression of PLXNA1 is associated with fast biochemical recurrence after radical prostatectomy through analysis on the Glinsky 2004 data and TCGA data (b, c).

FIG. 7 shows that, high expression of PLXNA1 in the two cohorts respectively of Chinese Prostate Cancer Alliance (a) and Massachusetts General Hospital (b) is closely related to biochemical recurrence, non-metastatic prostate cancer survival, and overall survival after radical surgery of prostate cancer in a patient; The proportion of patients with metastasis prostate cancer with increased PLXNA1 copy number was significantly higher than that of non-metastatic prostate cancer (c); the relationship between PLXNA1 gene copy number and biochemical recurrence after surgery was verified with the TCGA database (d).

### DETAILED DESCRIPTION OF THE INVENTION

The present invention will be further described in detail in conjunction with the following specific embodiments and drawings. The processes, conditions, experimental methods, and the like of the present invention are generally known in the art and common general knowledge, except for the contents specifically mentioned below, and the present invention is not particularly limited.

### Example 1 Expression of PLXNA1 at the Cellular Level

### (1.1) Effect of Si-RNA-Mediated PLXNA1 Knockout on Cell Phenotype

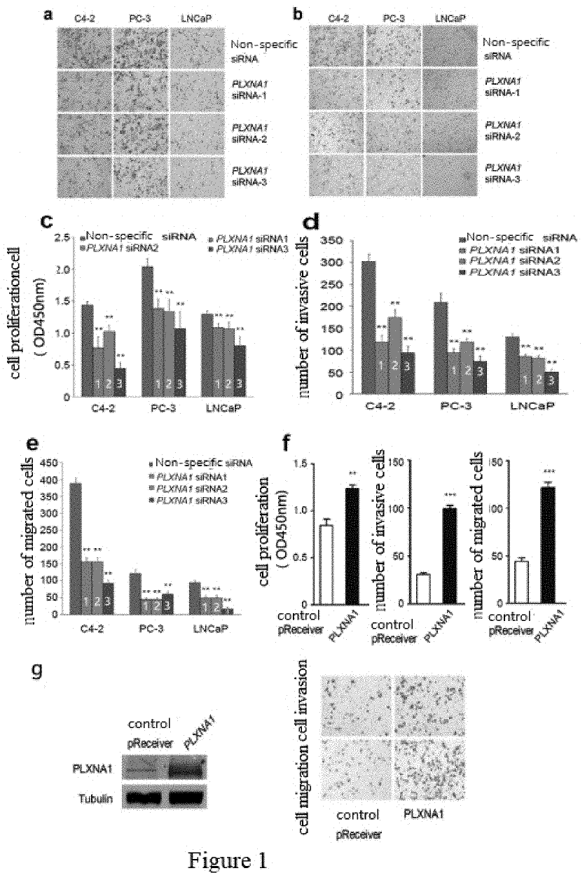

The present invention detected the effect of si-RNA-mediated PLXNA1 knockdown on cell phenotype and found that the knockdown of PLXNA1 by si-RNA significantly suppressed the invasive ability (FIG. 1a) and migration ability (FIG. 1b) of prostate cancer cells. In the different types of prostate cancer cell lines tested (including C4-2, PC-3, LNCaP), knockdown of the expression of PLXNA1 by si-RNA significantly reduced the abilities of cell proliferation, invasion and migration (FIG. 1c-e).

The prostate cancer cell lines involved in the experiment were all derived from the American ATCC cell line, and the cells were cultured in RPMI 1640+10% fetal bovine serum and placed in a 5% CO₂ incubator at 37° C.

Prior to the invasion and migration experiments, cells were plated into 6-well plates; 100 nM siRNA was transfected with Lipofectamine RNAiMAX (Life), and 6 hours later, normal medium was changed for invasion and migration experiments. The medium cell volume in each well was respectively C4-2 (6×10⁴per well), PC-3 (6×10⁴per well) and (LNCaP 5×10⁴per well), and the invasion experiment was performed with Matrigel-coated 8-μm Transwell inserts (BD), migration experiments was performed with 8μm Pore Size Transwell chamber (Costar). During the experiment, the upper chamber was 200 ul of serum-free cell suspension, and the lower layer was 500 ul of normal medium. The whole culture was cultured for 24 hours or 48 hours according to the instructions. After staining with crystal violet, 10 random fields of invasion and migration chamber were selected, and the cells are counted to measure the abilities of invasion and migration of prostate cancer cells.

### (1.2) Effects of Overexpression of PLXNA1 on Cell Proliferation, Invasion and Migration

In the prostate cancer cell line DU145, overexpression of PLXNA1 significantly promoted cell proliferation (FIG. 1f), invasion, and migration (FIG. 1g). In the overexpression experiment, cells were plated in 6-well plates and transfected with Invitrogen's transfection reagent Lipofectamine 2000, in which the amounts of plasmid in both the experimental and control groups were all 4 ug. After 6 hours of transfection, the medium was changed to normal medium for invasion and migration experiments, and the experimental method was the same as (1.1).

### (1.3) Effect of High Expression of PLXNA1 on Proliferation, Invasion and Migration of Progressive Prostate Cancer Cells

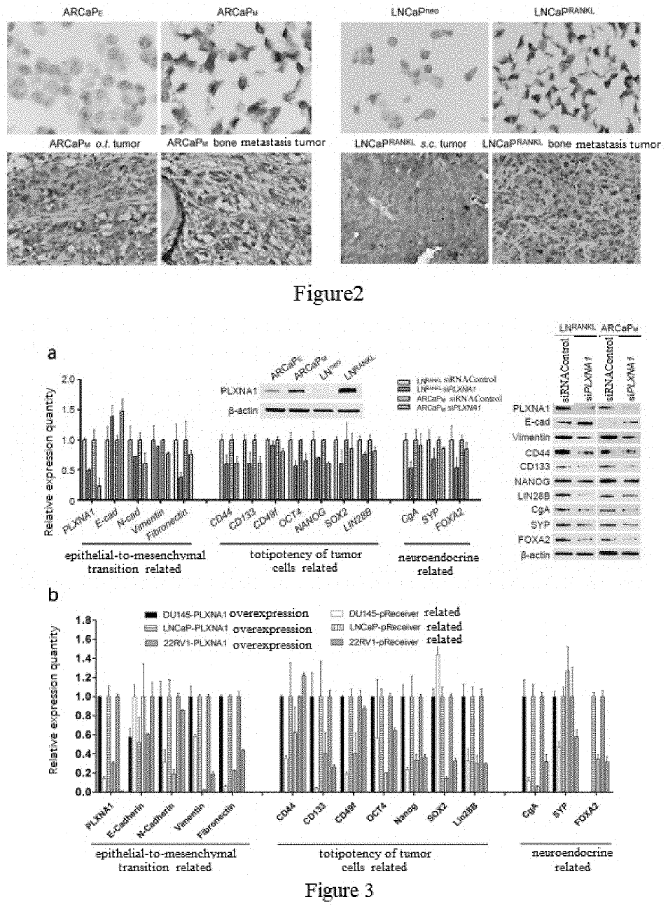

The same phenomenon was observed in the progressive prostate prostate cancer cell lines with high expression of PLXNA1 (ARCaP_{M} cell line and LNCaP^{RANKL} cell line) (FIG. 2), where proliferation, invasion, and migration of the cells were enhanced. Both cell lines have the properties of interstitial cell and are easy to transfer to bones and soft tissues (FIG. 2).

### (1.4) Correlation between Knockdown of PLXNA1 and Expression of Progressive Phenotypic Markers

The phenotype of epithelial-to-mesenchymal transition, totipotency and neuroendocrine properties are all associated with metastatic castration-resistant prostate cancer. The inventors of this invention made the hypothesis that knockdown of PLXNA1 may affect the expression of these progressive phenotypic markers. The present invention detected the change of the expression of relevant markers by q-RTPCR. After performing knockdown of PLXNA1 in LNCaP^{RANKL} cell line and ARCaPM cell line, the expression of interstitial cell marker vimentin, N-cadherin and fibronectin decreased, while the expression of epithelial cell marker E-cadherin increased (FIG. 3a). Moreover, it was observed that the expression of the stem cell markers CD44, CD133, CD49f, OCT4, NANOG, SOX2 and LIN28B and neuroendocrine cell markers CgA, SYP and FOXA2 were suppressed (FIG. 3a). Similarly, in the prostate cancer cell lines with abnormally high expression of PLXNA1, the epithelial-to-mesenchymal transition, totipotency, and neuroendocrine phenotype increased, indicating that the higher the degree of the tumor malignancy, the easier of recurrence, metastasis, and drug resistance (FIG. 3b).

### Example 2 Evaluation of the Effect of Knockdown of PLXNA1 on Cell Proliferation in Tumor-Bearing Nude Mice Model

### (2.1) Effect of ShRNA-Mediated Knockdown of PLXNA1 on Tumor Size and Weight

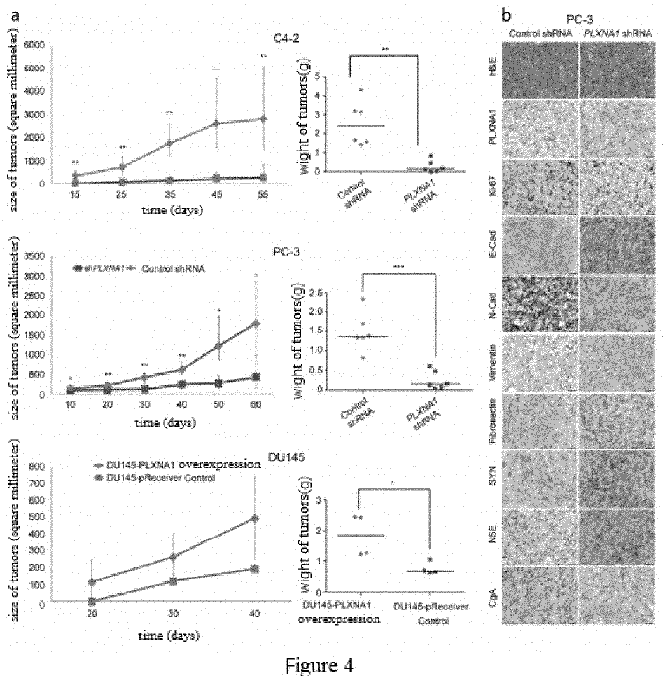

The effect of knockdown of PLXNA1 on cell growth was assessed in vivo with a tumor-bearing nude mouse model. Tumor size and weight in nude mice with shRNA-mediated PLXNA1 stable knockdown were significantly reduced than that in control group (FIG. 4a).

### (2.2) Effect of Overexpression of PLXNA1 on Tumor Growth of Tumor-Bearing Nude Mice

In the present invention, the tumor cells were subcutaneously injected into the nude mice of the control group or the experimental group by subcutaneous injection, and the proliferation of the tumor cells was observed. It was found that overexpression of PLXNA1 significantly promoted the tumor growth in the tumor-bearing nude mice.

Specifically, the proliferation of the prostate cancer cell line with overexpressing PLXNA1 in tumor-bearing mice was significantly faster than that of the control group (FIG. 4a).

### (2.3) Histopathological and Immunohistochemical Analysis after Knockdown of PLXNA1

Immunohistochemical staining analysis of the tumor in tumor-bearing mice showed that, the expression of Ki-67 (one of the markers of cell proliferation) decreased significantly after PLXNA1 knockdown, indicating that cell proliferation rate decreased significantly (FIG. 4b). In addition, compared with control group, phenotype reversal of the epithelial-to-mesenchymal transition was observed in the tumors with PLXNA1 knockdown, accompanied with the results of: increased expression of E-cadherin (E-cad) expression, increased expression of N-cadherin (N-cad), vimentin and fibronectin; along with suppressed expression of neuroendocrine phenotype, accompanied with the results of: weakened staining of CgA and Syp (FIG. 4b). The above was consistent with the phenomenon observed in malignant prostate cancer cell lines (FIG. 2).

Therefore, through the above relevant functional experiments, it was found that PLXNA1 plays an important role in the regulation of cell proliferation, invasion and metastasis, suggesting that high expression of PLXNA1 is associated with high degree of malignancy of tumor, and a change in the expression of PLXNA1 may be used to predict the occurrence, development, and prognosis of prostate cancer.

The immunohistochemistry staining assay mainly included the following steps:

1. Sectioning: conventional sectioning, 3-micron thickness pathology sections, placed on a gelled white piece.

2. Baking slices: Leica roasting machine, 75° C. for 30 minutes.

3. Dewaxing and hydrating: dewaxing with conventional xylene (three times, 5-10 mins each time), gradual ethanol hydration; washing with PBS for 5 mins*3 times after hydration.

4. Antigen retrieval: Antigen retrieval using alkali repair (EDTA, Fuzhou Maixin MVS-0099, 50X), with 1× repair solution. Using microwave heat repair method, first boiling in microwave oven on high was performed for 6 mins, then the section was placed in boiling repair solution, on medium and small, kept for 20 min.

5. Cool naturally at room temperature.

6. Elimination of endogenous peroxidase: using 3% H₂O₂ hydrogen peroxide to eliminate endogenous peroxidase, 15 min, then using PBS for washing 5 mins*3 times.

7. Secondary antibody homologous serum incubation: blocked with sheep serum, room temperature for 1 h.

8. Primary antibody incubation: incubated overnight (about 14 h) at 4° C.

9. Secondary antibody incubation: after incubation with primary antibody, washing with PBS for 5 mins*3 times, biotinylated goat anti-rabbit secondary antibody for 30 mins at room temperature; after incubation, washing with PBS for 5 mins*3 times.

10. Streptomycin-labeled HRP incubation: Streptavidin-HRP incubation for 15 min (Fuzhou Maixin), and then washing with PBS for 5 mins*3 times.

11. DAB staining: Fuzhou Maixin DAB coloring liquid (DAB-2031), after the staining, tap water is used to rinse to terminate the reaction.

12. Hematoxylin counterstaining: Hematoxylin was heated to 50° C., dyed for about 15 s, rinsed with tap water.

13. Hydrochloric acid differentiation: differentiated with 1% hydrochloric acid alcohol (1 ml concentrated hydrochloric acid, 100 ml absolute ethanol), and then rinsed with tap water.

14. Conventional dehydration, transparent, sealing: gradient ethanol dehydration, xylene transparent, neutral gum sealing.

### Example 3 Clinical Significance of PLXNA1 in the Evaluation of Occurrence, Development, and Prognosis of Prostate Cancer

### (3.1) Expression of PLXNA1 in Metastatic Prostate Cancer

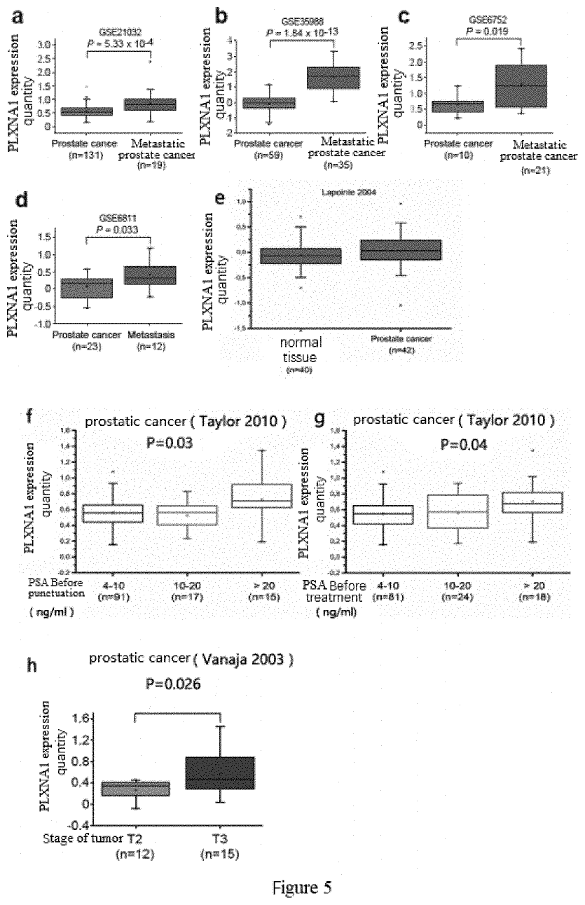

In order to evaluate the clinical significance of PLXNA1 in the occurrence, development of human prostate cancer, the present invention first studied and explored the possible relationship between the expression of PLXNA1 and the degree of malignancy of tumors. Dy data analysis on the existing clinical database, the inventors found that the expression of PLXNA1 was significantly higher in metastatic prostate cancer than in non-metastatic prostate cancer (FIG. 5a-d), and the expression of PLXNA1 was significantly higher in prostate cancer tissues than in the corresponding normal tissues (FIG. 5e).

### (3.2) Correlation between Expression of PLXNA1 and Cancer Prognosis

### (3.2.1) Correlation between PLXNA1 and PSA Level and Clinical Stage

In the present invention, prostate cancer patients were divided into different groups according to their PSA (Prostate specific antigen) level, by the criteria of PSA being 4-10, 10-20, >20, and it was found that the expression level of PLXNA1 increased with the increase of PSA level (FIG. 5f, g). According to the tumor staging of AJCC (American Joint Committee on Cancer), the present invention defined the two stages of prostate cancer at T2 stage and T3 stage. It was found that in the higher T3 stage prostate cancer, the expression level of PLXNA1 was significantly increased, indicating the expression of PLXNA1 is related to high clinical stage (FIG. 5h).

### (3.2.2) Correlation between Expression of PLXNA1 in Prostate Cancer Tissue and Prognosis of Prostate Cancer

To further clarify the relationship between the expression of PLXNA1 in prostate cancer tissues and prognosis of prostate cancer, the present invention detected the expression of PLXNA1 in a plurality of samples by immunohistochemistry staining (methods are the same as the aforementioned 2.3 immunohistochemistry method), and the relationship between the expression of PLXNA1 and the prognosis of patients was studied by combining the analysis of the prognostic data of patients,.

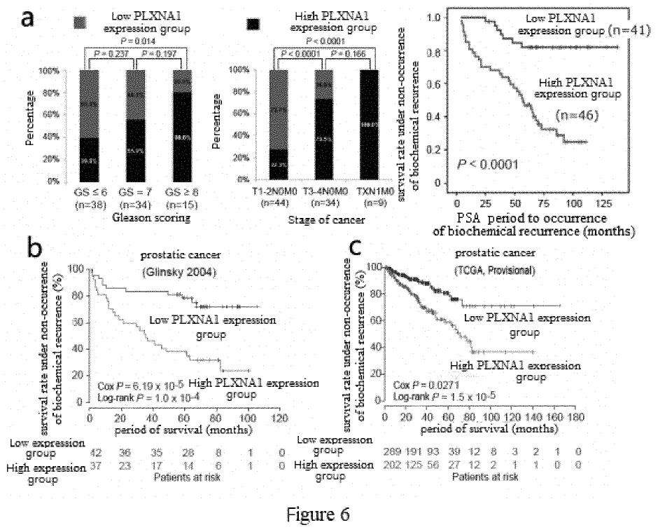

Through immunostaining of PLXNA1 in a separate cohort of 87 patients with primary prostate cancer from Shanghai Changhai Hospital, it was found that high expression of PLXNA1 was associated with high Gleason score, progressive tumor stage, and fast biochemical recurrence after radical surgery (FIG. 6a). The present studies showed that increased expression of PLXNA1 is associated with poor prognosis. In the other two independent cohorts of prostate cancer patients (data respectively from Glinsky 2004 and TCGA), the inventors of this invention also observed that high expression of PLXNA1 mRNA was associated with fast biochemical recurrence after radical surgery of prostate cancer in patients (FIG. 6b, c).

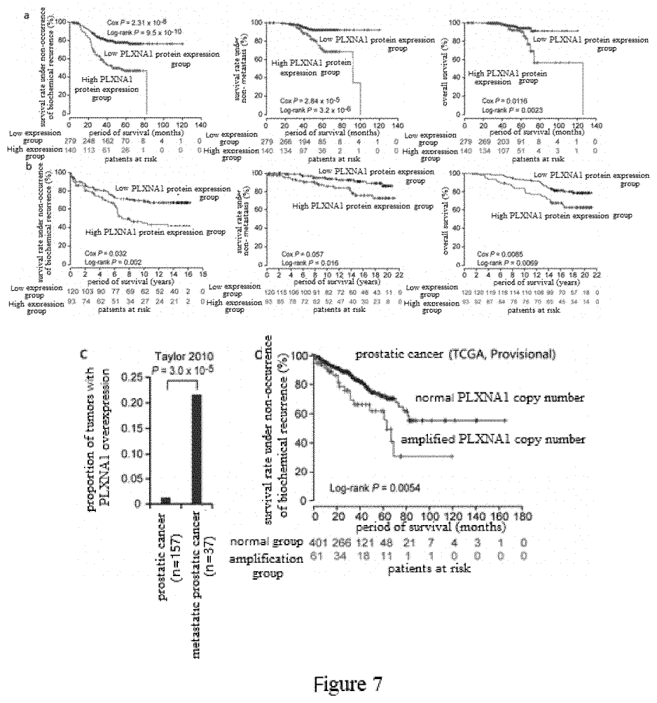

To further clarify the role of PLXNA1 in prognosis of prostate cancer, immunostaining of PLXNA1 was conducted by immunohistochemistry assay to detect the expression of PLXNA1 in two groups of independent samples of tissue microarray (one group from China Prostate Cancer Consortium (CPCC), n=419, 4 institutions, 10-year follow-up; another group from Massachusetts General Hospital (MGH), n=213, 20-year follow-up). It was found that high expression of PLXNA1 was not only associated with biochemical recurrence but also significantly with non-metastasis survival and overall survival (FIG. 7a, b). It is worth noting that multivariate and univariate regression analysis showed that increased expression of PLXNA1 was an independent factor predicting biochemical recurrence, non-metastasis survival and overall survival, prognosis in patients with prostate cancer (The risk ratios of CPCC group in patients with high expression of PLXNA1 for biochemical recurrence, non-metastasis survival, and overall survival were 2.75, 3.63, and 2.66 respectively. The risk ratios of Massachusetts General Hospital group in patients with high expression of PLXNA1 for biochemical recurrence, non-metastasis survival, and overall survival were 1.67, 2.31 and 2.24 respectively) (FIG. 7a, b). The above experimental data and analysis results suggest that PLXNA1 can be used as a novel biomarker to evaluate the degree of malignancy of prostate cancer.

### Example 4 Correlation between PLXNA1 Copy Number Variation and Prognosis of Prostate Cancer Patients

Since the increase in the expression of PLXNA1 is related to the copy number variation of DNA, as mentioned above, the increase in the expression of PLXNA1 is associated with a higher degree of malignancy of prostate cancer. The present inventors further investigated whether the increase in the copy number of PLXNA1 is directly related to the recurrence of prostate cancer.

Analysis showed that the frequency of increase in PLXNA1 copy number was very high in metastatic prostate cancer (FIG. 7c). Furthermore, by analyzing TCGA data of a cohort of prostate cancers patients, the inventors found that the probability of biochemical recurrence in patients with prostate cancer with increased PLXNA1 copy number was significantly higher than that in patients without increased PLXNA1 copy number (TCGA data, FIG. 7d).

Therefore, amplification of copy number and overexpression of PLXNA1 DNA can promote the growth and progression of prostate cancer, and they are associated with higher malignancy and poor prognosis of prostate cancer, and therefore can be used as a biomarker for prognosis of prostate cancer.

The protection of the present invention is not limited to the above embodiment. Any variations and advantages that may be conceived by those skilled in the art are intended to be included within the scope of the invention, and the scope of protection is attached by the appended claims.

What is claimed:

1. A prostate cancer biomarker PLXNA1, wherein the nucleotide sequence of the PLXNA1 is shown as SEQ ID No. 1.

2. A prostate cancer biomarker PLXNA1, wherein the protein sequence of PLXNA1 is shown as SEQ ID No. 2.

3. Use of a reagent for detecting of the biomarker PLXNA1 according to claim 1 in the preparation of a product for predicting the occurrence, development, and prognosis of prostate cancer.

4. Use of a reagent for detecting of the biomarker PLXNA1 according to claim 1 in the preparation of a product for distinguishing prostate cancer being progressive and with high malignancy.

5. An in vitro diagnostic product for prostate cancer, wherein the in vitro diagnostic product comprises a reagent for specific detection of PLXNA1 DNA, and/or a reagent for specific detection of PLXNA1 mRNA, and/or a reagent for specific detection of protein encoded by the PLXNA1 gene.

6. The in vitro diagnostic product for prostate cancer according to claim 5, wherein the in vitro diagnostic product comprises kit, gene chip, or solid support; the solid support includes arrays, microarrays, or protein arrays.

7. Use of the PLXNA1 according to claim 1 in the preparation of pharmaceuticals for suppressing the proliferation, metastasis, invasion of prostate cancer cells, and for suppressing the growth, metastasis of prostate tumors.

8. Use of the PLXNA1 according to claim 1 in the preparation of pharmaceuticals for suppressing the expression of interstitial cell marker vimentin, N-cadherin, fibronectin, stem cell marker and neuroendocrine cell marker.

9. Use of the PLXNA1 according to claim 1 in the preparation of pharmaceuticals for promoting the expression of epithelial cell marker E-cadherin.

10. Use of an inhibitor of the PLXNA1 according to claim 1 in the preparation of pharmaceuticals for treating prostate cancer.

11. A method for predicting the occurrence, development, and prognosis of prostate cancer. distinguishing the degree of malignancy of progressive prostate cancer in patients who are diagnosed with prostate cancer comprising the following steps:
a) detecting the DNA copy numbers of PLXNA1, or the expression levels of mRNA or the protein encoded thereby in pathology samples of prostate cancer;
b) dividing the patients into a high expression group and a low expression group by referring to the results of the previous step a);
if DNA copy number of PLXNA1 is amplified or overexpressed of the patients compared to normal population, or if the expression level of mRNA or the protein encoded thereby of the patient increases, then the patient is classified into high expression group, indicating deteriorating, highly invasive of tumor, poor prognosis for survival, likelihood of biochemical recurrence, distant metastasis or disease progression or death; whereas the patients is classified into low expression group.


SEQUENCE LISTING (2 sequences)

SEQ ID NO: 1 - DNA, 9071 bp, Artificial Sequence
  Synthetic PLXNA1 mRNA
atgccgctgccaccgcggagcctgcaggtgctcctgctgctgctgctgttgctgctgctgctgccgggcatgtgggctgaggcaggcttgcccagggcaggcgggggttcacagccccccttccgcaccttctcggccagcgactggggcctcacccacctagtggtgcatgagcagacaggcgaggtgtatgtgggcgcagtgaaccgcatctataagctgtcggggaacctgacactgctgcgggcccacgtcacgggccctgtggaggacaacgagaagtgctacccgccgcccagcgtgcagtcctgcccccacggcctgggcagtactgacaacgtcaacaagctgctgctgctggactatgccgctaaccgcctgctggcctgtggcagcgcctcccagggcatctgccagttcctgcgtctggacgatctcttcaaactgggtgagccacaccaccgtaaggagcactacctgtccagcgtgcaggaggcaggcagcatggcgggcgtgctcattgccgggccaccgggccagggccaggccaagctcttcgtgggcacacccatcgatggcaagtccgagtacttccccacactgtccagccgtcggctcatggccaacgaggaggatgccgacatgttcggcttcgtgtaccaggatgagtttgtgtcatcacagctcaagatcccttcggacacgctgtccaagttcccggcctttgacatctactatgtgtacagcttccgcagcgagcagtttgtctactacctcacgctgcagctagacacacagctgacctcgcctgatgccgccggcgagcacttcttcacgtccaagatcgtgcggctctgtgtggacgaccccaaattctactcgtacgttgagttccccattggctgcgagcaggcgggtgtggagtaccgcctggtgcaggatgcctacctgagccggcccggccgtgccctggcccaccagctgggcctggctgaggacgaggacgtgctgttcactgtgttcgcccagggccagaagaaccgcgtgaagccaccaaaggagtcagcactgtgcctgttcacgctcagggccatcaaggagaagattaaggagcgcatccagtcctgctaccgtggtgagggcaagctctccctgccgtggctgctcaacaaggagctgggctgcatcaactcgcccctgcagatcgatgacgacttctgcgggcaggacttcaaccagcccctggggggcacagtcaccattgaggggacgcccctgttcgtggacaaggatgatggcctgaccgccgtggctgcctatgactatcggggccgcactgtggtattcgccggcacgcgaagtggccgcatccgcaagatcctggtggacctctcaaaccccggtggccggcctgccctggcctacgagagcgtcgtggcccaggagggcagccccatcctgcgagacctcgtcctcagccccaaccaccagtacctctacgccatgaccgagaagcaggtgacgcgggtgcctgtggagagctgtgtgcagtacacgtcctgtgagctgtgtctggggtcacgggacccccactgtggctggtgtgtcctgcacagcatctgctcgcggcgggacgcctgtgagcgagcagacgagccccagcgctttgctgcggacctgctgcagtgtgtgcagctgactgtgcagccccgcaatgtgtctgtcaccatgtcccaggtcccacttgtgctgcaggcctggaacgtgcctgacctctcagctggcgtcaactgctccttcgaggacttcacggaatctgagagcgtcctggaggatggccggatccactgccgctcaccctccgcccgggaggtggcgcccatcacgcggggccagggagaccagcgggtggtgaaactctacctaaagtccaaggagacagggaagaagtttgcgtctgtggacttcgtcttctacaactgcagcgtccaccagtcctgcctgtcctgtgtcaacggctcctttccctgccactggtgcaaataccgccacgtgtgcacacacaacgtggctgactgcgccttcctggagggccgtgtcaacgtgtctgaggactgcccacagatcctgccctccacgcagatctacgtgccagtgggagtggtaaaacccatcaccctggccgcacggaacctgccacagccacagtcaggccagcgtggatatgagtgcctcttccacatcccgggcagcccggcccgtgtcaccgccctgcgcttcaacagctccagcctgcagtgccagaattcctcgtactcctacgaggggaacgatgtcagcgacctgccagtgaacctgtcagtcgtgtggaacggcaactttgtcattgacaacccacagaacatccaggcgcacctctacaagtgcccggccctgcgcgagagctgcggcctctgcctcaaggccgacccgcgcttcgagtgcggatggtgcgtggccgagcgccgctgctccctgcgacaccactgcgctgccgacacacctgcatcgtggatgcacgcgcgtcacggcagcagtcgctgcaccgaccccaagatcctcaagctgtcccccgagacgggcccgaggcagggcggcacgcggctcactatcacaggcgagaacctgggcctgcgattcgaagacgtgcgtctgggcgtgcgcgtgggcaaggtgctgtgcagccctgtggagagcgagtacatcagtgcggagcagatcgtctgtgagatcggggacgccagctccgtgcgtgcccatgacgccctggtggaggtgtgtgtgcgggactgctcaccacactaccgcgccctgtcacccaagcgcttcaccttcgtgacaccaaccttctaccgtgtgagcccctcccgtgggcctctgtcagggggcacctggattggcatcgagggaagccacctgaacgcaggcagtgatgtggctgtgtcggtcggtggccggccctgctccttctcctggaggaactcccgtgagatccggtgcctgacaccccccgggcagagccctggcagcgctcccatcatcatcaacatcaaccgcgcccagctcaccaaccctgaggtgaagtacaactacaccgaggaccccaccatcctgaggatcgaccccgagtggagcatcaacagcggtgggaccctcctgacggtcacaggcaccaacctggccactgtccgtgaaccccgaatccgggccaagtatggaggcattgagagggagaacggctgcctggtgtacaatgacaccaccatggtatgccgcgccccgtctgtggccaaccctgtgcgcagcccaccagagctgggggagcggccggatgagctgggcttcgtcatggacaacgtgcgctccctgcttgtgctcaactccacctccttcctctactaccctgaccccgtactggagccactcagccccactggcctgctggagctgaagcccagctccccactcatcctcaagggccggaacctcttgccacctgcacccggcaactcccgactcaactacacggtgctcatcggctccacaccctgtaccctcaccgtgtcggagacgcaactgctgtgcgaggcgcccaacctcactgggcagcacaaggtcacggtgcgggcaggtggcttcgagttctcgccagggacactgcaggtgtactcggacagcctgctgacgctgcctgccattgtgggcattggcggaggcgggggtctcctgctgctggtcatcgtggctgtgctcatcgcctacaagcgcaagtcacgagatgctgaccgcacactcaagcggctgcagctccagatggacaacctggagtcccgcgtggccctcgaatgcaaggaagcctttgcagagctgcagacagacatccacgagctgaccaatgacctggacggtgccggcatccccttccttgactaccggacatatgccatgcgggtgctctttcctgggatcgaggaccaccctgtgctcaaggagatggaggtgcaggccaatgtggagaagtcgctgacactgttcgggcagctgctgaccaagaagcacttcctgctgaccttcatccgcacgctggaggcacagcgcagcttctccatgcgcgaccgcgggaatgtggcctcgctcatcatgacggccctgcagggcgagatggaatacgccacaggcgtgctcaagcagctgctttccgacctcatcgagaagaacctggagagcaagaaccaccccaagctgctactgcgccggactgagtcggtggcagagaagatgctaactaactggttcaccttcctcttgtataagttcctcaaggagtgcgctggggagccgctgttcatgctgtactgcgccatcaagcagcagatggagaagggccccattgacgccatcacgggtgaggcacgctactccctgagtgaggacaagctcatccggcagcagattgactacaagacactgaccctgaactgtgtgaaccctgagaatgagaatgcacctgaggtgccggtgaaggggctggactgtgacacggtcacccaggccaaggagaagctgctggacgctgcctacaagggcgtgccctactcccagcggcccaaggccgcggacatggacctggagtggcgccagggccgcatggcgcgcatcatcctgcaggacgaggacgtcaccaccaagattgacaacgattggaagaggctgaacacactggctcactaccaggtgacagacgggtcctcggtggcactggtgcccaagcagacgtccgcctacaacatctccaactcctccaccttcaccaagtccctcagcagatacgagagcatgctgcgcacggccagcagccccgacagcctgcgctcgcgcacgcccatgatcacgcccgacctggagagcggcaccaagctgtggcacctggtgaagaaccacgaccacctggaccagcgtgagggtgaccgcggcagcaagatggtctcggagatctacttgacacggctactggccaccaagggcacactgcagaagtttgtggacgacctgtttgagaccatcttcagcacggcacaccggggctcagccctgccgctggccatcaagtacatgttcgacttcctggatgagcaggccgacaagcaccagatccacgatgctgacgtgcgccacacctggaagagcaactgcctgcccctgcgcttctgggtgaacgtgatcaagaacccacagtttgtgttcgacattcacaagaacagcatcacggacgcctgcttgtcggtggtggcccagaccttcatggactcctgctccacctctgagcacaagctgggcaaggactcaccctccaacaagctgctctacgccaaggacatccccaactacaagagctgggtggagaggtactatgcagacatcgccaagatgccagccatcagcgaccaggacatgagtgcgtatctggctgagcagtcccgcctgcacctgagccagttcaacagcatgagcgccttgcacgagatctactcctacatcaccaagtacaaggatgagatcctggcagccctggagaaggatgagcaggcgcggcggcagcggctgcggagcaagctggagcaggtggtggacacgatggccctgagcagctgagccccagctgtgatcatccagcatgatgcagcgtgaggacagctgagcagggaccgggacagccctcaccgcatgcgtgtggagtgtccggtggtgctcgggccgccgcagtgcagcgactgcccggccctccctcccctgcctcacccggtcgggtcccggctcttcctgtgtggaggtgatggtacctgccacaccacagctgcgcacacagctgcttgctcaggggccgggacagcactgggtgctcaggctggccaaggaccttcattgcctggcaagagctgcccagtggccttcatgggagaagggctgacctctgaggggctgaggggtgaggccagggccctccagggggaggggtagccagcttgggctgtccccttgagaccaggacaagaggctgggggtgtcagcattcccagctttccaagctgcccccaggcggcagagtctgagggtcccggggcccggttggcagctggagaaagaggcaaaaagcccgtagccgggcaagaggagctcaagtcggtctgggcccgttgccaccgactcccacctccagcacccatgcccgctgcaccgctgccatcctcagattcaccgcgtgctctgcgcggccgaggccggagcaccacatccacctcgccccagagaggctctgctccctcctatggaggggctgtgggccaggctgctcagactcctgggtggcttccagacggaccgggcagcccctctccgtcctcagggctgtgcctctgggagccactgggccaggggccccgggtcgcagagagcacgttcccgttatttattcccctccgcgtcctacacaggctgccctggcagctgtcttcaagggtaggctgagctccccaccctggagcccctgagggcggcccctgagcactcctctctctccactctctctgtccctgccccagcggcttccagtgtggcatctcagcagtgtcctggcccctccagagcagtgggacatctggggactgtttttgtgtttaggggaaaaaattctgctgcactctgcttgggccttgaggtctgtggcagggctcctctggcccgcagtggcctggatctatctgggccatgagtgacgggcagtgaccagagggactggaggccagcggtgtccacccttgccctcagcaagagagaatgcattcttaaaagaaagctgtacatgtatatatatgcatatatatatatgtggctctagcctcaggctccagccccagtggggtactgtacagttaactgaagaagaattttaaagacgatttgaacaagaaaatgaaggcagtgggaaagcaatgccaaatggttgtggagaaagtggccggagcctccctggagtggagcagccctgaagcctgtgccccccgacctgcgggccgctgttttggtttgacatgacaaggaaaggacttcctgctgaccctgagagcctctggggtgccgcggcaccacggggcatgcatgattgtgctagcgtttagtctgagttgatctttttaaaactgcaagtgttgaatactagaggttgttagacccttttttatgttttttaattaatcagtcacttgtaaaagcaaacaagcggtccatccccttttcaaggtcacttttttgatggtaccgaagatcccatggacattaagggacagctaactgtggccagactcagccccatgtccttggccaggcccaaggagaggactcggccccatggggtgtgccagtcttgcagtccgccccagctgagtagcgtgagccagatgacgccacagagacccgcctcttccctgaacgcgggtcggtgtggagtcagtgactgctgactcagggagctccttggccccgtgggcactgtgccagggctggggccttctgctgctgccacacccagctcaggcctgggccagcccctgcccccagcccactgagggggtgggcttactccctgggcagtcttgggggccagagctgaggccagtccatattacagtggctgggctgtttttttcagtagcccctagcattggctgggattcctgttcctgggtgcgcctccacctcccttctgatgtttcctggctatggtggggtgggaacctcagtttcccccaaagtcttccctggatgctggcttcaggttgaagtccctggttcttccagttcctcacgggttaggtaggggctcctgcatcaccttcagaatccagttccaacccccactctccttaggccttgtgctctgctctgccctgccaggctgcccttgtccatgtgagtagcatgggcgggtggtggggacggcagtggtgatgaagggggtgcaccacaggcctcatgaagcagttcccacatgggcgtgtggctggggcgtggccaccacagagcacatggctgtgtctaggcgcaagcactttagcagtatctgtttacatgcgcaaggatcaagccgactacctgtgctgtctactgggacagcagtctccgagctactccgtacctccctctgccaggtcgtggagttaggccccagtccctacttgtcactggttcccactgtgctcctaactgtgcagcacctgggagctctggcctggggctggaggccctggtaggagctgcagttggaggccgttctgtgcccagcagcggtgagcggctcccatgggccctgtgtctgcagggagccagggctgcggcacatgtgctgtgaaactggcacccacctggcgtgctgctgccgccacttgcttcctgcagcacctcctaccctgctccgtgtcctccctctccccgcgcctggctcaggagtgctggaaaagctcacgcctcggcctgggagcctggcctcttgatatacctcgagcttcccctgtgctccccagccccaggaccactggccccttggcctgaggggctgggggccccacgacctgcagcgtcgagtccgggagagagcccggagcggcgtgccatctcggctcggccttgctgagagcctccgccctggctttctccctgtctggattcagtggctcacgttggtgctacacagctagaatagatatatttagagagagagatatttttaagacaaagcccacaattagctgtcctttaacaccgcagaaccccctcccagaagaagagcgatccctcggacggtccgggcgggcaccctcagccgggctctttgcagaagcagcaccgctgactgtgggcccggccctcagatgtgtacatatacggctatttcctattttactgttcttcagatttagtacttgtaaataaacacacacattaaggagagattaaacatttttgctaaaagctaaaaa

SEQ ID NO: 2 - protein, 1896 aa, Artificial Sequence
  Synthetic PLXNA1 protein
MPLPPRSLQVLLLLLLLLLLLPGMWAEAGLPRAGGGSQPPFRTFSASDWGLTHLVVHEQTGEVYVGAVNRIYKLSGNLTLLRAHVTGPVEDNEKCYPPPSVQSCPHGLGSTDNVNKLLLLDYAANRLLACGSASQGICQFLRLDDLFKLGEPHHRKEHYLSSVQEAGSMAGVLIAGPPGQGQAKLFVGTPIDGKSEYFPTLSSRRLMANEEDADMFGFVYQDEFVSSQLKIPSDTLSKFPAFDIYYVYSFRSEQFVYYLTLQLDTQLTSPDAAGEHFFTSKIVRLCVDDPKFYSYVEFPIGCEQAGVEYRLVQDAYLSRPGRALAHQLGLAEDEDVLFTVFAQGQKNRVKPPKESALCLFTLRAIKEKIKERIQSCYRGEGKLSLPWLLNKELGCINSPLQIDDDFCGQDFNQPLGGTVTIEGTPLFVDKDDGLTAVAAYDYRGRTVVFAGTRSGRIRKILVDLSNPGGRPALAYESVVAQEGSPILRDLVLSPNHQYLYAMTEKQVTRVPVESCVQYTSCELCLGSRDPHCGWCVLHSICSRRDACERADEPQRFAADLLQCVQLTVQPRNVSVTMSQVPLVLQAWNVPDLSAGVNCSFEDFTESESVLEDGRIHCRSPSAREVAPITRGQGDQRVVKLYLKSKETGKKFASVDFVFYNCSVHQSCLSCVNGSFPCHWCKYRHVCTHNVADCAFLEGRVNVSEDCPQILPSTQIYVPVGVVKPITLAARNLPQPQSGQRGYECLFHIPGSPARVTALRFNSSSLQCQNSSYSYEGNDVSDLPVNLSVVWNGNFVIDNPQNIQAHLYKCPALRESCGLCLKADPRFECGWCVAERRCSLRHHCAADTPASWMHARHGSSRCTDPKILKLSPETGPRQGGTRLTITGENLGLRFEDVRLGVRVGKVLCSPVESEYISAEQIVCEIGDASSVRAHDALVEVCVRDCSPHYRALSPKRFTFVTPTFYRVSPSRGPLSGGTWIGIEGSHLNAGSDVAVSVGGRPCSFSWRNSREIRCLTPPGQSPGSAPIIININRAQLTNPEVKYNYTEDPTILRIDPEWSINSGGTLLTVTGTNLATVREPRIRAKYGGIERENGCLVYNDTTMVCRAPSVANPVRSPPELGERPDELGFVMDNVRSLLVLNSTSFLYYPDPVLEPLSPTGLLELKPSSPLILKGRNLLPPAPGNSRLNYTVLIGSTPCTLTVSETQLLCEAPNLTGQHKVTVRAGGFEFSPGTLQVYSDSLLTLPAIVGIGGGGGLLLLVIVAVLIAYKRKSRDADRTLKRLQLQMDNLESRVALECKEAFAELQTDIHELTNDLDGAGIPFLDYRTYAMRVLFPGIEDHPVLKEMEVQANVEKSLTLFGQLLTKKHFLLTFIRTLEAQRSFSMRDRGNVASLIMTALQGEMEYATGVLKQLLSDLIEKNLESKNHPKLLLRRTESVAEKMLTNWFTFLLYKFLKECAGEPLFMLYCAIKQQMEKGPIDAITGEARYSLSEDKLIRQQIDYKTLTLNCVNPENENAPEVPVKGLDCDTVTQAKEKLLDAAYKGVPYSQRPKAADMDLEWRQGRMARIILQDEDVTTKIDNDWKRLNTLAHYQVTDGSSVALVPKQTSAYNISNSSTFTKSLSRYESMLRTASSPDSLRSRTPMITPDLESGTKLWHLVKNHDHLDQREGDRGSKMVSEIYLTRLLATKGTLQKFVDDLFETIFSTAHRGSALPLAIKYMFDFLDEQADKHQIHDADVRHTWKSNCLPLRFWVNVIKNPQFVFDIHKNSITDACLSVVAQTFMDSCSTSEHKLGKDSPSNKLLYAKDIPNYKSWVERYYADIAKMPAISDQDMSAYLAEQSRLHLSQFNSMSALHEIYSYITKYKDEILAALEKDEQARRQRLRSKLEQVVDTMALSS